(19) United States
(12) Patent Application Publication
(10) Pub. No.: US 2023/0398154 A1
(43) Pub. Date: Dec. 14, 2023

(54) A COMPOSITION COMPRISING MESENCHYMAL PRECURSOR OR STEM CELLS AND THEIR USE

(71) Applicant: Mesoblast International Sárl, Meyrin (CH)

(72) Inventors: Silviu ITESCU, Melbourne (AU); Paul SIMMONS, Melbourne (AU)

(21) Appl. No.: 18/041,303

(22) PCT Filed: Aug. 9, 2021
(86) PCT No.: PCT/IB2021/057314

§ 371 Date: Feb. 10, 2023

(30) Foreign Application Priority Data
Aug. 10, 2020 (AU) 2020902827

(51) Int. Cl. A61K 35/28; A61P 37/06; G01N 33/68; C12N 5/0775; A61P 29/00

(52) U.S. Cl. CPC A61K 35/28; A61P 37/06; G01N 33/6863; C12N 5/0663; A61P 29/00; G01N 2333/7151; G01N 2800/245

(57) ABSTRACT

The present disclosure relates to improved cellular compositions and potency assays for obtaining the same. Such compositions and assays may be suitable for use in treating various inflammatory disorders.

### TECHNICAL FIELD

The present disclosure relates to improved cellular compositions and potency assays for obtaining the same. Such compositions and assays may be suitable for use in treating various inflammatory disorders.

### BACKGROUND

Several cellular therapy products for regenerative or immune therapy applications have advanced to clinical evaluation and market authorization. However, release of these cellular therapy products onto the market is hindered by their complexity and heterogeneity, which makes identification of relevant biologic activities and thus, definition of consistent cellular therapy product quality difficult.

Physiochemical parameters (for example, characterization of size, morphology, light-scattering properties, tensile strength, cell number, confluence, identification of phenotypic markers, secreted substances, genotype, gene expression profile) are routinely used for identification and quantification of the active substance, intermediates, impurities and contaminants. However, physiochemical parameters cannot confirm that a product will be biologically active and potent (i.e., elicit the desired effect). In contrast, biologic characterization takes into account the effect of the product on biologic systems, either modelled in vitro or in vivo in animals and ultimately in the clinic.

Potency testing must demonstrate the relevant biologic activity or activities of the product. It is not a requirement for potency testing to reflect all of the product's biological functions, but it should indicate one or more relevant biological functions. It is expected that accuracy, sensitivity, specificity and reproducibility will be established for the analytic methods used in potency testing and that they be suitably robust.

There is a need to develop products with improved potency for treatment of diseases where immunosuppression is desired. It is also preferable to identify parameters that are critical to the efficacy of cellular therapy products and to control them (e.g., via potency testing) such that products of consistent quality can be manufactured.

### SUMMARY

Preparations of mesenchymal precursor lineage or stem cells (MLPSC)s have now been identified with enhanced therapeutic potential. Such preparations express increased levels of receptor(s) which direct activation of NF NF-κB (i.e. NF-κB phosphorylation) when exposed to inflammatory stimuli such as those stimuli associated with various inflammatory diseases. Accordingly, in an example, the present disclosure relates to a composition comprising a population of culture expanded mesenchymal lineage precursor or stem cells (MLPSC), wherein the population of MLPSCs are culture expanded from a cryopreserved intermediate MLPSC preparation and the culture expanded MLPSCs express a receptor which, upon activation via an inflammatory stimulus, phosphorylates NF-κB, wherein the MLPSCs express a level of the receptor that is sufficient to increase phosphorylation of NF-κB upon activation via the inflammatory stimulus at least 3.5 fold greater than unstimulated MLPSCs. In an example, the MLPSCs express a level of the receptor that is sufficient to increase phosphorylation of NF-κB upon activation via the inflammatory stimulus at least 4 fold greater than unstimulated MLPSCs. In an example, the receptor is one or both of TNF-R1 or IL-1R. In an example, activation of the MLPSCs via the inflammatory stimulus increases secretion of one or more of MCP-1, M-CSF and PGE2 under culture conditions. For example, activation of the MLPSCs via the inflammatory stimulus may result in secretion of one or more of the following under culture conditions: 18,000 pg/ml MCP-1; 1,500 pg/ml M-CSF; 40,000 pg/ml PGE2.

In an example, the composition comprises at least 25×10⁶ cells.

In another example, the phosphorylation of NF-κB is determined after contacting the cells with TNF-α and/or IL-1β. In this example, unstimulated MLPSCs are not contacted with TNF-α and/or IL-1β.

In an example, the receptor is TNF-R1.

In an example, compositions of the disclosure express high levels of TNF-R1 and improve therapeutic outcomes in inflammatory disease, particularly in relation to patient survival. Furthermore, MLPSCs in these preparations can be expanded to provide a therapeutic dose; even clinical scale preparations can now be made. Accordingly, in an example, the present disclosure encompasses a composition comprising a population of culture expanded mesenchymal lineage precursor or stem cells (MLPSC), wherein the population of MLPSCs are culture expanded from a cryopreserved intermediate MLPSC preparation and the culture expanded MLPSCs express at least about 200 pg/ml TNF-R1 under culture conditions. For example, the culture expanded MLPSCs can express at least about 23.5 pg TNF-R1 per 10⁶ cells under culture conditions. In another example, the present disclosure encompasses a composition comprising a population of culture expanded mesenchymal lineage precursor or stem cells (MLPSC), wherein the population of MLPSCs are culture expanded from a cryopreserved intermediate MLPSC preparation and the culture expanded MLPSCs express at least about 225 pg/ml TNF-R1 under culture conditions. For example, the culture expanded MLPSCs can express at least about 26.5 pg TNF-R1 per 10⁶ cells under culture conditions. In another example, the culture expanded MLPSCs express at least about 230 pg/ml TNF-R1 under culture conditions. For example, the culture expanded MLPSCs can express at least about 27 pg TNF-R1 per 10⁶ cells under culture conditions. In an example, In an example, the composition comprises at least 25×10⁶ cells. Accordingly, in another example, the present disclosure encompasses a composition comprising a population of culture expanded mesenchymal lineage precursor or stem cells, wherein the composition comprises at least 25×10⁶ cells and, wherein the population of culture expanded cells has been selected for use in treatment of an inflammatory disease by determining expression of least about 200 pg/ml TNF-R1 under culture conditions. In an example, the population of culture expanded cells has been selected for use in treatment of an inflammatory disease by determining expression of least about 220 pg/ml TNF-R1 under culture conditions. In an example, the population of culture expanded cells has been selected for use in treatment of an inflammatory disease by determining expression of least about 230 pg/ml TNF-R1 under culture conditions. In another example, the population of culture expanded cells has been selected for use in treatment of an inflammatory disease by determining expression of least about 25×10⁶ cells TNF-R1 under culture conditions.

In an example, the population of culture expanded cells are culture expanded in a bioreactor. In another example, the inflammatory disease is graft versus host disease (GvHD).

In an example, the composition comprises a cryopreservative.

In another example, the MLPSCs inhibit IL-2Rα expression by at least 55% under culture conditions. In another example, the MLPSCs inhibit IL-2Rα expression by at least 58% under culture conditions. In another example, the MLPSCs inhibit IL-2Rα expression by at least 60% under culture conditions. In another example, the MLPSCs inhibit IL-2Rα expression between 55 and 75% under culture conditions. In another example, the MLPSCs inhibit IL-2Rα expression between 58 and 65% under culture conditions.

In another example, the present disclosure encompasses a composition comprising a population of culture expanded mesenchymal lineage precursor or stem cells (MLPSC), wherein the population of MLPSCs are culture expanded from a cryopreserved intermediate MLPSC preparation and the culture expanded MLPSCs inhibit IL-2Rα expression by at least 55% under culture conditions. In another example, the MLPSCs inhibit IL-2Rα expression by at least 58% under culture conditions. In another example, the MLPSCs inhibit IL-2Rα expression by at least 60% under culture conditions. In another example, the MLPSCs inhibit IL-2Rα expression between 55 and 75% under culture conditions. In another example, the MLPSCs inhibit IL-2Rα expression between 58 and 65% under culture conditions.

In another embodiment, the present inventors have developed a potency assay to measure the biological activity or therapeutic efficacy of cellular therapy products comprising mesenchymal lineage precursor or stem cells. Accordingly, in another example, the present disclosure relates to a method for determining the therapeutic efficacy of mesenchymal lineage precursor or stem cells comprising:
(i) obtaining a population comprising mesenchymal lineage precursor or stem cells;
(ii) culturing the cells in a culture medium; and
(iii) determining the amount of TNF-R1 expressed by the cells into the culture medium under culture conditions, wherein an amount of at least about 200 pg/ml TNF-R1 is indicative of therapeutic efficacy.

In an example, part (iii) comprises determining the amount of TNF-R1 expressed by the cells into the culture medium under culture conditions, wherein an amount of at least about 225 pg/ml TNF-R1 is indicative of therapeutic efficacy.

In an example, part (iii) comprises determining the amount of TNF-R1 expressed by the cells into the culture medium under culture conditions, wherein an amount of at least about 230 pg/ml TNF-R1 is indicative of therapeutic efficacy.

In an example, the population of mesenchymal lineage precursor or stem cells is obtained from a 3D cell culture.

In an example, the population of mesenchymal lineage precursor or stem cells are culture expanded in 3D cell culture from a cryopreserved intermediate MLPSC preparation.

In another example, the present disclosure relates to a method of treating a subject with an inflammatory disease, the method comprising administering to a subject in need thereof a composition of mesenchymal lineage precursor or stem cells, wherein the cells express at least about 200 pg/ml TNF-R1 under culture conditions. In an example, the cells express at least about 225 pg/ml TNF-R1 under culture conditions. In an example, the cells express at least about 230 pg/ml TNF-R1 under culture conditions.

In an example, the inflammatory disease is a T cell mediated inflammatory disease. In another example, the inflammatory disease is GvHD. In an example, the GvHD is chronic GvHD. In an example, a treated subject has improved 100 day survival.

In an example, the subject is refractory to steroid immunosuppressant and/or a biologic therapy. In an example, the subject receives at least two doses of the composition. In an example, the subject receives twice weekly dosing.

In an example, the mesenchymal precursor lineage or stem cells are mesenchymal stem cells. In an example, the composition further comprises Plasma-Lyte A, dimethyl sulfoxide (DMSO), human serum albumin (HSA). In an example, the composition comprises Plasma-Lyte A (70%), DMSO (10%), HSA (25%) solution, the HSA solution comprising 5% HSA and 15% buffer. In another example, each dose comprises at least 2×10⁶ cells/kg body weight of subject.

### BRIEF DESCRIPTION OF DRAWINGS

FIG. 1: (Upper LHS): siRNA-mediated knockdown of TNF-R1 in MSCs. Culture expanded MSC were transfected with siRNA targeting TNF-R1 (20, 100 or 500 pM) for 4 h. (Lower LHS): Phosphorylated NF-kB in unstimulated and TNFα-stimulated MSCs. OD=optical density. Columns represent mean±SD. (Upper RHS) CCL2 secretion from MSCs following siRNA-mediated knockdown of TNF-R1 and contact with TNFα. (Lower RHS) M-CSF secretion from MSCs following siRNA-mediated knockdown of TNF-R1 and contact with TNFα.

FIG. 2: TNFα induces expression of immunomodulatory cytokines regulated by NF-kB in culture expanded MSCs. Bars represent mean±SD.

FIG. 3: Effect of blocking antibody to MCP-1 on TNFa mediated production of IL-10 by CD14+ monocytes co-cultured with MSCs.

FIG. 4: Product manufactured with improved process has higher mean TNF-R1 levels and less variability in inhibition of IL-2Rα.

FIG. 5: Manufacturing changes resulting in increased TNF-R1 levels have been accompanied by increased Day 100 survival.

FIG. 6: Significantly improved survival in patients that received MSCs associated with IL-2Rα expression levels >60%.

FIG. 7: IL2Ra inhibition in vitro correlates with in vivo reduction of Activated CD4+ T cells.

### DETAILED DESCRIPTION

### General Techniques and Definitions

Unless specifically defined otherwise, all technical and scientific terms used herein shall be taken to have the same meaning as commonly understood by one of ordinary skill in the art (e.g., in cell culture, molecular biology, stem cell culture, immunology, and biochemistry).

Unless otherwise indicated, cell culture techniques and assays utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

As used herein, the term “about”, unless stated to the contrary, refers to +/−10%, more preferably +/−5%, of the designated value.

The terms “level” and “amount” are used to define the amount of a particular substance in a cell preparation. For example, a particular concentration, weight, percentage (e.g. v/v %) or ratio can be used to define the level of a particular substance. In an example, the level is expressed in terms of how much of a particular marker is expressed by cells of the disclosure under culture conditions. In an example, expression represents cell surface expression. In another example, the level is expressed in terms of how much of a particular marker is release from cells described herein under culture conditions.

In an example, the level is expressed in pg/ml. In another example, the level is expressed in pg per 10⁶ cells. The level of pg/ml can be converted to pg per 10⁶ cells if required. For example, in the context of TNF-R1, in an example, 200 pg/ml TNF-R1 corresponds to about 23.5 pg of TNF-R1 per 10⁶ cells. In an example, in the context of TNF-R1, in an example, 225 pg/ml TNF-R1 corresponds to about 26.5 pg of TNF-R1 per 10⁶ cells. In an example, 230 pg/ml TNF-R1 corresponds to about 27 pg of TNF-R1 per 10⁶ cells. In another example, 260 pg/ml TNF-R1 corresponds to about 30 pg of TNF-R1 per 10⁶ cells. In another example, 270 pg/ml TNF-R1 corresponds to about 32 pg TNF-R1 per 10⁶ cells and so on.

In an example, the level of a particular marker is determined under culture conditions. The term “culture conditions” is used to refer to cells growing in culture. In an example, culture conditions refers to an actively dividing population of cells. Such cells may, in an example, in exponential growth phase. For example, the level of a particular marker can be determined by taking a sample of cell culture media and measuring the level of marker in the sample. In another example, the level of a particular marker can be determined by taking a sample of cells and measuring the level of the marker in the cell lysate. Those of skill in the art that secreted markers will be measured by sampling the culture media while markers expressed on the surface of the cell may be measured by assessing a sample of cell lysate. In an example, the sample is taken when the cells are in exponential growth phase. In an example, the sample is taken after at least two days in culture.

Culture expanding cells from a cryopreserved intermediate means thawing cells subject to cryogenic freezing and in vitro culturing under conditions suitable for growth of the cells.

In an example, the “level” or “amount” of a particular marker such as TNF-R1 is determined after cells have been cryopreserved and then seeded back into culture. For example, the level is determined after a first cryopreservation of cells. In another example, the level is determined after a second cryopreservation of cells. For example, cells may be culture expanded from a cryopreserved intermediate, cryopreserved a second time before being re-seeded in culture so that the level of a particular marker can be determined under culture conditions.

In an example, the cells express a receptor which is activated by an inflammatory stimulus. Such receptors bind to inflammatory mediators such as cytokines. Various examples of such receptors are known and include, for example, TNF-R1 and IL-1R. Those of skill in the art will appreciate that an appropriate inflammatory stimulus will be dictated by the receptor being targeted. For example, an appropriate inflammatory stimulus for TNF-R1 is TNF-alpha. In another example, an appropriate inflammatory stimulus for IL-1R is IL-1.

The term “seeding” is used herein to refer to the process of introducing cells into 3 dimensional (3D) culture. In an example, the cells of the disclosure are seeded into culture via dynamic seeding, wherein the culture medium continues to mix as cells attach to adherent material. In an another example, the cells are seeded into 3D culture and left for a period of time sufficient to adhere to the adherent material in the culture medium, such that the cells can attach to the material.

In an example, mesenchymal lineage precursor or stem cells are seeded at between 5,000 and 20,000 cells/ml. In another example, mesenchymal lineage precursor or stem cells are seeded at between 8,000 and 20,000 cells/ml. In another example, mesenchymal lineage precursor or stem cells are seeded at between 8,000 and 15,000 cells/ml.

The term “recovering” is used herein to refer to removing cells from 2D or 3D culture. For example, cells can be recovered from a culture disclosed herein. In an example, recovered cells are first washed (e.g. 2-3 times) with a saline solution or comparable solution. Subsequent to the washing step, a dissociating step may be conducted on the adherent material. In one example, a suitable dissociation enzyme is employed during the dissociating step. In an example, cells recovered from 3D culture are washed and concentrated before being cryopreserved. In an example, the washed and concentrated cells can be stored, filled, finished and visually inspected before being cryopreserved.

As used herein, the terms “treating”, “treat” or “treatment” include administering a population of mesenchymal lineage stem or precursor cells and/or progeny thereof and/or soluble factors derived therefrom to thereby reduce or eliminate at least one symptom of a disease disclosed herein. In an example, treatment includes administering a composition of the disclosure. In an example, the treatment induces a partial response after treatment is initiated. In an example, the treatment induces a complete response after treatment is initiated.

In the context of treating GvHD, in an example, the partial response is induced 28 days after treatment is initiated. In an example, the partial response is induced at least 30 days after treatment is initiated. In an example, the partial response is induced at least 2 months after treatment is initiated. In another example, the partial response is induced at least 3 months after treatment is initiated. In another example, the partial response is induced 28 to 56 days after treatment is initiated. In another example, the partial response is induced after two doses. In another example, the partial response is induced after two doses administered once weekly. In another example, the partial response is induced after two doses administered once weekly every two weeks. In another example, the partial response is induced after three doses or more.

In an example, a partial response in GvHD is characterized by one or more or all of:
Reduction in Skin % BSA score of at least one point;
Reduction in mouth score of at least one point;
Reduction in eye score of at least one point;
Reduction in skin features score of at least one point;
Reduction in gastrointestinal tract score of at least one point;
Reduction in liver score of at least one point;
Reduction in lung symptom score of at least one point;
Reduction in lung FEV1 score of at least one point;
Reduction in joints and fascia score of at least one point;
Reduction in genital tract score of at least one point.

In an example, a partial response is characterized by a reduction in Skin % BSA score of at least one point. In another example, a partial response is characterized by a reduction in mouth score of at least one point. In another example, a partial response is characterized by a reduction in eye score of at least one point. In these examples, scores can be obtained using the NIH Consensus Criteria 2014 for GvHD (see for example, the Examples section below).

In an example, compositions of the disclosure may be administered to prevent or inhibit GvHD. The term “prevent” or “preventing” as used herein include administering a population of mesenchymal lineage stem or precursor cells and/or progeny thereof and/or soluble factors derived therefrom to thereby stop or inhibit the development of at least one symptom of cGvHD.

There are various classification systems for characterizing GvHD (Lee, S., (2017) Blood, 129(1): 30-37). In an example, the NIH Consensus Criteria 2014 can be used for scoring outcomes disclosed herein (Jagasia et al., (2015) Biol Blood Marrow Transplant, 21:389-401). The components of the NIH Consensus Criteria 2014 are shown in the following table:

Organ Scoring of cGvHD

| Organ | Score 0 | Score 1 | Score 2 | Score 3 |
|---|---|---|---|---|
| Skin % BSA¹ | No BSA involved | 1-18% BSA | 19-50% BSA | >50% BSA |
| Skin Features | No sclerotic | N/A | Superficial sclerotic | Deep sclerotic features; |
| | features | | features, but not | “hidebound”; impaired |
| | | | “hidebound” | mobility; ulceration |
| Mouth | No symptoms | Mild symptoms | Moderate symptoms | Severe symptoms with |
| | | with disease | with disease signs with | disease signs with |
| | | signs but not | partial limitation of oral | major limitation of oral |
| | | limiting oral | intake | intake |
| | | intake | | |
| | | significantly | | |
| Eyes | No symptoms | Mild dry eye | Moderate dry eye | Severe dry eye |
| | | symptoms not | symptoms partially | symptoms significantly |
| | | affecting ADL | affecting ADL (requiring | affecting ADL (special |
| | | (requirement of | lubricant drops > 3×/day | eyewear to relieve pain) |
| | | lubricant | or punctal plugs) | OR unable to work |
| | | drops ≤ 3×/day) | WITHOUT new vision | because of ocular |
| | | | impairment due to | symptoms OR loss of |
| | | | keratoconjunctivitis | vision due to |
| | | | sicca (KCS) | keratoconjunctivitis |
| | | | | sicca (KCS) |
| GI Tract | No symptoms | Symptoms | Symptoms associated | Symptoms associated |
| | | without | with mild to moderate | with significant weight |
| | | significant | weight loss (5-15%) | loss (>15%) within 3 |
| | | weight loss | within 3 months OR | months, requires |
| | | (<5%) | moderate diarrhea | nutritional supplement |
| | | | without significant | for most calorie needs |
| | | | interference with daily | OR esophageal dilation |
| | | | living | OR severe diarrhea with |
| | | | | significant interference |
| | | | | with daily living |
| Liver | Normal total | Normal total | Elevated total bilirubin | Elevated total bilirubin |
| | bilirubin and | bilirubin with | but ≤3 mg/dl or ALT > | but >3 mg/dL |
| | ALT or AP < 3 × | ALT ≥ 3 to 5 × | 5 × ULN | |
| | ULN | ULN or AP ≥ 3 × | | |
| | | ULN | | |
| Lungs | No symptoms | Mild symptoms | Moderate symptoms | Severe symptoms (SOB |
| Symptom | | (SOB after | (SOB after walking on | at rests; requires O2) |
| Score | | climbing one | flat ground) | |
| | | flight of steps) | | |
| Lungs Lung | FEV1 ≥ 80% | FEV1 60-79% | FEV1 40-59% | FEV1 ≤ 39% |
| Score | | | | |
| Joints and | No symptoms | Mild tightness of | Tightness of arms or legs | Contractures WITH |
| Fascia | | arms and legs, | OR joint contractures, | significant decrease of |
| | | normal or mild | erythema thought to be | range of motion AND |
| | | decreased range | due to fasciitis, | significant limitation of |
| | | of motion AND | moderate decrease of | ADL (unable to tie |
| | | not affecting | range of motion AND | shoes, button shirts, |
| | | ADL | mild to moderate | dress shelf, etc.) |
| | | | limitation of ADL | |
| Genital Tract2 | No signs | Mild signs and | Moderate signs and may | Severe signs with or |
| | | females with or | have signs of discomfort | without symptoms |
| | | without | on exam | |
| | | discomfort on | | |
| | | exam | | |
| Other | No GVHD | Mild | Moderate | Severe |
| features3 | | | | |

Again, in the context of GvHD, in an example, a partial response is a decrease of ≥1 point on the organ-specific NIH Consensus Criteria 2014 score from the Table above. Accordingly, in an example, treatment induces ≥1 point decrease in Skin % BSA score. In another example, treatment induces ≥1 point decrease in mouth score. In another example, treatment induces ≥1 point decrease in eye score. In another example, treatment induces ≥1 point decrease in skin features score. In another example, treatment induces ≥1 point decrease in gastrointestinal tract score. In another example, treatment induces ≥1 point decrease in liver score. In another example, treatment induces ≥1 point decrease in lung symptom score. In another example, treatment induces ≥1 point decrease in lung FEV1 score. In another example, treatment induces ≥1 point decrease in joints and fascia score. In another example, treatment induces ≥1 point decrease in genital tract score.

In an example, the treatment induces a complete response for GvHD after treatment is initiated. In an example, the complete response is induced 28 days after treatment is initiated. In an example, the complete response is induced at least 28 after treatment is initiated. In an example, the complete response is induced at least 30 after treatment is initiated. In an example, the complete response is induced at least 2 months after treatment is initiated. In another example, the complete response is induced at least 3 months after treatment is initiated. In another example, the complete response is induced 28 to 56 days after treatment is initiated. In another example, the complete response is induced after two doses. In another example, the complete response is induced after two doses administered once weekly. In another example, the complete response is induced after two doses administered once weekly every two weeks. In another example, the complete response is induced after three doses or more.

In an example, methods of the present disclosure inhibit cGvHD disease progression or disease complication in a subject. “Inhibition” of cGvHD disease progression or disease complication in a subject means preventing or reducing cGvHD progression and/or disease complication in the subject.

In an example, treatment increases patient survival. In an example, treatment increases the probability of a subject surviving for at least 100 days after initiation of treatment. In an example, the increased probability is determined relative to a subject that is not treated with a composition of the disclosure.

The term “inflammatory bowel disease” (IBD) is used in the context of the present disclosure refer to inflammatory diseases of the gastrointestinal tract such as ulcerative colitis (UC), irritable bowel syndrome, irritable colon syndrome Crohn's colitis and Crohn's disease (CD).

The term “ulcerative colitis (UC)” can include mild-to-moderate ulcerative colitis. Mild-to-moderate ulcerative colitis can be characterized by one or, more or all of the following:
A score of 4-10 on the ulcerative colitis-disease activity index (UC-DAT);
A sigmoidoscopy score of >4;
A Physician's Global Assessment (PGA) score of >2).

The term “subject” as used herein refers to a human subject. For example, the subject can be an adult. In another example, the subject can be a child. In another example, the subject can be an adolescent. Terms such as “subject”, “patient” or “individual” are terms that can, in context, be used interchangeably in the present disclosure. In an example, the subject is refractory to steroid therapy. In an example, the subject is refractory to a biologic therapy. In an example, the subject is refractory to steroid therapy and a biologic therapy.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Those skilled in the art will appreciate that the disclosure described herein is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific embodiments described herein, which are intended for the purpose of exemplification only. Functionally-equivalent products, compositions and methods are clearly within the scope of the disclosure, as described herein.

Any example disclosed herein shall be taken to apply mutatis mutandis to any other example unless specifically stated otherwise.

In an example, compositions of the disclosure comprise genetically unmodified mesenchymal precursor lineage or stem cells. As used herein, the term “genetically unmodified” refers to cells that have not been modified by transfection with a nucleic acid. For the avoidance of doubt, in the context of the present disclosure a mesenchymal lineage precursor or stem cell transfected with a nucleic acid encoding TNF-R1 would be considered genetically modified.

### Mesenchymal Lineage Precursor Cells

As used herein, the term “mesenchymal lineage precursor or stem cell (MLPSC)” refers to undifferentiated multipotent cells that have the capacity to self-renew while maintaining multipotency and the capacity to differentiate into a number of cell types either of mesenchymal origin, for example, osteoblasts, chondrocytes, adipocytes, stromal cells, fibroblasts and tendons, or non-mesodermal origin, for example, hepatocytes, neural cells and epithelial cells. For the avoidance of doubt, a “mesenchymal lineage precursor cell” refers to a cell which can differentiate into a mesenchymal cell such as bone, cartilage, muscle and fat cells, and fibrous connective tissue.

The term “mesenchymal lineage precursor or stem cells” includes both parent cells and their undifferentiated progeny. The term also includes mesenchymal precursor cells, multipotent stromal cells, mesenchymal stem cells (MSCs), perivascular mesenchymal precursor cells, and their undifferentiated progeny.

Mesenchymal lineage precursor or stem cells can be autologous, allogeneic, xenogenic, syngenic or isogenic. Autologous cells are isolated from the same individual to which they will be reimplanted. Allogeneic cells are isolated from a donor of the same species. Xenogenic cells are isolated from a donor of another species. Syngenic or isogenic cells are isolated from genetically identical organisms, such as twins, clones, or highly inbred research animal models.

In an example, the mesenchymal lineage precursor or stem cells are allogeneic. In an example, the allogeneic mesenchymal lineage precursor or stem cells are culture expanded and cryopreserved.

Mesenchymal lineage precursor or stem cells reside primarily in the bone marrow, but have also shown to be present in diverse host tissues including, for example, cord blood and umbilical cord, adult peripheral blood, adipose tissue, trabecular bone and dental pulp. They are also found in skin, spleen, pancreas, brain, kidney, liver, heart, retina, brain, hair follicles, intestine, lung, lymph node, thymus, ligament, tendon, skeletal muscle, dermis, and periosteum; and are capable of differentiating into germ lines such as mesoderm and/or endoderm and/or ectoderm. Thus, mesenchymal lineage precursor or stem cells are capable of differentiating into a large number of cell types including, but not limited to, adipose, osseous, cartilaginous, elastic, muscular, and fibrous connective tissues. The specific lineage-commitment and differentiation pathway which these cells enter depends upon various influences from mechanical influences and/or endogenous bioactive factors, such as growth factors, cytokines, and/or local microenvironmental conditions established by host tissues.

The terms “enriched”, “enrichment” or variations thereof are used herein to describe a population of cells in which the proportion of one particular cell type or the proportion of a number of particular cell types is increased when compared with an untreated population of the cells (e.g., cells in their native environment). In one example, a population enriched for mesenchymal lineage precursor or stem cells comprises at least about 0.1% or 0.5% or 1% or 2% or 5% or 10% or 15% or 20% or 25% or 30% or 50% or 75% mesenchymal lineage precursor or stem cells. In this regard, the term “population of cells enriched for mesenchymal lineage precursor or stem cells” will be taken to provide explicit support for the term “population of cells comprising X % mesenchymal lineage precursor or stem cells”, wherein X % is a percentage as recited herein. The mesenchymal lineage precursor or stem cells can, in some examples, form clonogenic colonies, e.g. CFU-F (fibroblasts) or a subset thereof (e.g., 50% or 60% or 70% or 70% or 90% or 95%) can have this activity.

In an example of the present disclosure, the mesenchymal lineage precursor or stem cells are mesenchymal stem cells (MSCs). The MSCs may be a homogeneous composition or may be a mixed cell population enriched in MSCs. Homogeneous MSC compositions may be obtained by culturing adherent marrow or periosteal cells, and the MSCs may be identified by specific cell surface markers which are identified with unique monoclonal antibodies. A method for obtaining a cell population enriched in MSCs is described, for example, in U.S. Pat. No. 5,486,359. Alternative sources for MSCs include, but are not limited to, blood, skin, cord blood, muscle, fat, bone, and perichondrium. In an example, the MSCs are allogeneic. In an example, the MSCs are cryopreserved. In an example, the MSCs are culture expanded and cryopreserved.

In another example, the mesenchymal lineage precursor or stem cells are CD29+, CD54+, CD73+, CD90+, CD102+, CD105+, CD106+, CD166+, MHC1+ MSCs.

Isolated or enriched mesenchymal lineage precursor or stem cells can be expanded in vitro by culture. Isolated or enriched mesenchymal lineage precursor or stem cells can be cryopreserved, thawed and subsequently expanded in vitro by culture.

In one example, isolated or enriched mesenchymal lineage precursor or stem cells are seeded at 50,000 viable cells/cm² in culture medium (serum free or serum-supplemented), for example, alpha minimum essential media (αMEM) supplemented with 5% fetal bovine serum (FBS) and glutamine, and allowed to adhere to the culture vessel overnight at 37° C., 20% O₂. The culture medium is subsequently replaced and/or altered as required and the cells cultured for a further 68 to 72 hours at 37° C., 5% O₂.

As will be appreciated by those of skill in the art, cultured mesenchymal lineage precursor or stem cells are phenotypically different to cells in vivo. For example, in one embodiment they express one or more of the following markers, CD44, NG2, DC146 and CD140b. Cultured mesenchymal lineage precursor or stem cells are also biologically different to cells in vivo, having a higher rate of proliferation compared to the largely non-cycling (quiescent) cells in vivo.

In one example, the population of cells is enriched from a cell preparation comprising STRO-1+ cells in a selectable form. In this regard, the term “selectable form” will be understood to mean that the cells express a marker (e.g., a cell surface marker) permitting selection of the STRO-1+ cells. The marker can be STRO-1, but need not be. For example, as described and/or exemplified herein, cells (e.g., mesenchymal precursor cells) expressing STRO-2 and/or STRO-3 (TNAP) and/or STRO-4 and/or VCAM-1 and/or CD146 and/or 3G5 also express STRO-1 (and can be STRO-1bright). Accordingly, an indication that cells are STRO-1+ does not mean that the cells are selected solely by STRO-1 expression. In one example, the cells are selected based on at least STRO-3 expression, e.g., they are STRO-3+(TNAP+).

Reference to selection of a cell or population thereof does not necessarily require selection from a specific tissue source. As described herein STRO-1+ cells can be selected from or isolated from or enriched from a large variety of sources. That said, in some examples, these terms provide support for selection from any tissue comprising STRO-1+ cells (e.g., mesenchymal precursor cells) or vascularized tissue or tissue comprising pericytes (e.g., STRO-1+ pericytes) or any one or more of the tissues recited herein.

In one example, the cells used in the present disclosure express one or more markers individually or collectively selected from the group consisting of TNAP+, VCAM-1+, THY-1+, STRO-2+, STRO-4+(HSP-90β), CD45+, CD146+, 3G5+ or any combination thereof.

By “individually” is meant that the disclosure encompasses the recited markers or groups of markers separately, and that, notwithstanding that individual markers or groups of markers may not be separately listed herein the accompanying claims may define such marker or groups of markers separately and divisibly from each other.

By “collectively” is meant that the disclosure encompasses any number or combination of the recited markers or groups of markers, and that, notwithstanding that such numbers or combinations of markers or groups of markers may not be specifically listed herein the accompanying claims may define such combinations or sub-combinations separately and divisibly from any other combination of markers or groups of markers.

As used herein the term “TNAP” is intended to encompass all isoforms of tissue non-specific alkaline phosphatase. For example, the term encompasses the liver isoform (LAP), the bone isoform (BAP) and the kidney isoform (KAP). In one example, the TNAP is BAP. In one example, TNAP as used herein refers to a molecule which can bind the STRO-3 antibody produced by the hybridoma cell line deposited with ATCC on 19 Dec. 2005 under the provisions of the Budapest Treaty under deposit accession number PTA-7282.

Furthermore, in one example, the STRO-1+ cells are capable of giving rise to clonogenic CFU-F.

In one example, a significant proportion of the STRO-1+ cells are capable of differentiation into at least two different germ lines. Non-limiting examples of the lineages to which the STRO-1+ cells may be committed include bone precursor cells; hepatocyte progenitors, which are multipotent for bile duct epithelial cells and hepatocytes; neural restricted cells, which can generate glial cell precursors that progress to oligodendrocytes and astrocytes; neuronal precursors that progress to neurons; precursors for cardiac muscle and cardiomyocytes, glucose-responsive insulin secreting pancreatic beta cell lines. Other lineages include, but are not limited to, odontoblasts, dentin-producing cells and chondrocytes, and precursor cells of the following: retinal pigment epithelial cells, fibroblasts, skin cells such as keratinocytes, dendritic cells, hair follicle cells, renal duct epithelial cells, smooth and skeletal muscle cells, testicular progenitors, vascular endothelial cells, tendon, ligament, cartilage, adipocyte, fibroblast, marrow stroma, cardiac muscle, smooth muscle, skeletal muscle, pericyte, vascular, epithelial, glial, neuronal, astrocyte and oligodendrocyte cells.

In an example, mesenchymal lineage precursor or stem cells are obtained from a single donor, or multiple donors where the donor samples or mesenchymal lineage precursor or stem cells are subsequently pooled and then culture expanded.

Mesenchymal lineage precursor or stem cells encompassed by the present disclosure may also be cryopreserved prior to administration to a subject. In an example, mesenchymal lineage precursor or stem cells are culture expanded and cryopreserved prior to administration to a subject.

In an example, the present disclosure encompasses mesenchymal lineage precursor or stem cells as well as progeny thereof, soluble factors derived therefrom, and/or extracellular vesicles isolated therefrom. In another example, the present disclosure encompasses mesenchymal lineage precursor or stem cells as well as extracellular vesicles isolated therefrom. For example, it is possible to culture expand mesenchymal precursor lineage or stem cells of the disclosure for a period of time and under conditions suitable for secretion of extracellular vesicles into the cell culture medium. Secreted extracellular vesicles can subsequently be obtained from the culture medium for use in therapy.

The term “extracellular vesicles” as used herein, refers to lipid particles naturally released from cells and ranging in size from about 30 nm to as a large as 10 microns, although typically they are less than 200 nm in size. They can contain proteins, nucleic acids, lipids, metabolites, or organelles from the releasing cells (e.g., mesenchymal stem cells; STRO-1⁺ cells).

The term “exosomes” as used herein, refers to a type of extracellular vesicle generally ranging in size from about 30 nm to about 150 nm and originating in the endosomal compartment of mammalian cells from which they are trafficked to the cell membrane and released. They may contain nucleic acids (e.g., RNA; microRNAs), proteins, lipids, and metabolites and function in intercellular communication by being secreted from one cell and taken up by other cells to deliver their cargo.

### Culture Expansion of the Cells

In an example, mesenchymal lineage precursor or stem cells are culture expanded. “Culture expanded” mesenchymal lineage precursor or stem cells media are distinguished from freshly isolated cells in that they have been cultured in cell culture medium and passaged (i.e. sub-cultured). In an example, culture expanded mesenchymal lineage precursor or stem cells are culture expanded for about 4-10 passages. In an example, mesenchymal lineage precursor or stem cells are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages. In these examples, stem cells may be culture expanded before being cryopreserved to provide an intermediate cryopreserved MLPSC population. In an example, compositions of the present disclosure are produced by culturing cells from an intermediate cryopreserved MLPSC population or, put another way, a cryopreserved intermediate.

In an example, compositions of the disclosure comprise mesenchymal lineage precursor or stem cells that are culture expanded from a cryopreserved intermediate. In an example, the cells culture expanded from a cryopreserved intermediate are culture expanded for at least 5, at least 6, at least 7, at least 8, at least 9, at least 10 passages. For example, mesenchymal lineage precursor or stem cells can be culture expanded for at least 5 passages. In an example, mesenchymal lineage precursor or stem cells can be culture expanded for at least 5-10 passages. In an example, mesenchymal lineage precursor or stem cells can be culture expanded for at least 5-8 passages. In an example, mesenchymal lineage precursor or stem cells can be culture expanded for at least 5-7 passages. In an example, mesenchymal lineage precursor or stem cells can be culture expanded for more than 10 passages. In another example, mesenchymal lineage precursor or stem cells can be culture expanded for more than 7 passages.

In an example, mesenchymal lineage precursor or stem cells culture expanded from a cryopreserved intermediate can be culture expanded in medium free of animal proteins. In an example, mesenchymal lineage precursor or stem cells culture expanded from a cryopreserved intermediate can be culture expanded in xeno-free medium. In an example, mesenchymal lineage precursor or stem cells culture expanded from a cryopreserved intermediate can be culture expanded in medium that is fetal bovine serum free.

In an embodiment, mesenchymal lineage precursor or stem cells can be obtained from a single donor, or multiple donors where the donor samples or mesenchymal lineage precursor or stem cells are subsequently pooled and then culture expanded. In an example, the culture expansion process comprises:
i. expanding by passage expansion the number of viable cells to provide a preparation of at least about 1 billion of the viable cells, wherein the passage expansion comprises establishing a primary culture of isolated mesenchymal lineage precursor or stem cells and then serially establishing a first non-primary (P1) culture of isolated mesenchymal lineage precursor or stem cells from the previous culture;
ii. expanding by passage expansion the P1 culture of isolated mesenchymal lineage precursor or stem cells to a second non-primary (P2) culture of mesenchymal lineage precursor or stem cells; and,
iii. preparing and cryopreserving an in-process intermediate mesenchymal lineage precursor or stem cells preparation obtained from the P2 culture of mesenchymal lineage precursor or stem cells; and,
iv. thawing the cryopreserved in-process intermediate mesenchymal lineage precursor or stem cells preparation and expanding by passage expansion the in-process intermediate mesenchymal lineage precursor or stem cells preparation.

In an example, the expanded mesenchymal lineage precursor or stem cell preparation has an antigen profile and an activity profile comprising:
i. less than about 0.75% CD45+ cells;
ii. at least about 95% CD105+ cells;
iii. at least about 95% CD166+ cells.

In an example, the expanded mesenchymal lineage precursor or stem cell preparation is capable of inhibiting IL2-Rα expression by CD3/CD28-activated PB MC s by at least about 30% relative to a control.

In an example, culture expanded mesenchymal lineage precursor or stem cells are culture expanded for about 4-10 passages, wherein the mesenchymal lineage precursor or stem cells have been cryopreserved after at least 2 or 3 passages before being further culture expanded. In an example, mesenchymal lineage precursor or stem cells are culture expanded for at least 1, at least 2, at least 3, at least 4, at least 5 passages, cryopreserved and then further culture expanded for at least 1, at least 2, at least 3, at least 4, at least 5 passages before being cultured according to the methods of the disclosure.

The process of mesenchymal lineage precursor or stem cell isolation and ex vivo expansion can be performed using any equipment and cell handing methods known in the art. Various culture expansion embodiments of the present disclosure employ steps that require manipulation of cells, for example, steps of seeding, feeding, dissociating an adherent culture, or washing. Any step of manipulating cells has the potential to insult the cells. Although mesenchymal lineage precursor or stem cells can generally withstand a certain amount of insult during preparation, cells are preferably manipulated by handling procedures and/or equipment that adequately performs the given step(s) while minimizing insult to the cells.

In an example, mesenchymal lineage precursor or stem cells are washed in an apparatus that includes a cell source bag, a wash solution bag, a recirculation wash bag, a spinning membrane filter having inlet and outlet ports, a filtrate bag, a mixing zone, an end product bag for the washed cells, and appropriate tubing, for example, as described in U.S. Pat. No. 6,251,295, which is hereby incorporated by reference.

In an example, a mesenchymal lineage precursor or stem cell composition cultured according to the present disclosure is 95% homogeneous with respect to being CD105 positive and CD166 positive and being CD45 negative. In an example, this homogeneity persists through ex vivo expansion; i.e. though multiple population doublings.

In an example, mesenchymal lineage precursor or stem cells of the disclosure are culture expanded in 3D culture. For example, mesenchymal lineage precursor or stem cells of the disclosure can be culture expanded in a bioreactor. In an example, mesenchymal lineage precursor or stem cells of the disclosure are initially culture expanded in 2D culture prior to being further expanded in 3D culture. In an example, mesenchymal lineage precursor or stem cells of the disclosure are culture expanded from a master cell bank. In an example, mesenchymal lineage precursor or stem cells of the disclosure are culture expanded from a master cell bank in 2D culture before seeding in 3D culture. In an example, mesenchymal lineage precursor or stem cells of the disclosure are culture expanded from a master cell bank in 2D culture for at least 3 days before seeding in 3D culture in a bioreactor. In an example, mesenchymal lineage precursor or stem cells of the disclosure are culture expanded from a master cell bank in 2D culture for at least 4 days before seeding in 3D culture in a bioreactor. In an example, mesenchymal lineage precursor or stem cells of the disclosure are culture expanded from a master cell bank in 2D culture for between 3 and 5 days before seeding in 3D culture in a bioreactor. In these examples, 2D culture can be performed in a cell factory. Various cell factory products are available commercially (e.g. Thermofisher, Sigma).

### Cell Culture Medium

Mesenchymal lineage precursor or stem cells disclosed herein can be culture expanded in various suitable growth mediums.

The term “medium” or “media” as used in the context of the present disclosure, includes the components of the environment surrounding the cells. The media contributes to and/or provides the conditions suitable to allow cells to grow. Media may be solid, liquid, gaseous or a mixture of phases and materials. Media can include liquid growth media as well as liquid media that do not sustain cell growth. Media also include gelatinous media such as agar, agarose, gelatin and collagen matrices. Exemplary gaseous media include the gaseous phase that cells growing on a petri dish or other solid or semisolid support are exposed to.

The cell culture media used for culture expansion contains all essential amino acids and may also contain non-essential amino acids. In general, amino acids are classified into essential amino acids (Thr, Met, Val, Leu, Ile, Phe, Trp, Lys, His) and non-essential amino acids (Gly, Ala, Ser, Cys, Gln, Asn, Asp, Tyr, Arg, Pro).

Those of skill in the art will appreciate that for optimal results, the basal medium must be appropriate for the cell line of interest. For example, it may be necessary to increase the level of glucose (or other energy source) in the basal medium, or to add glucose (or other energy source) during the course of culture, if this energy source is found to be depleted and to thus limit growth. In an example, dissolved oxygen (DO) levels can also be controlled.

In an example, the cell culture medium contains human derived additives. For example, human serum and human platelet cell lysate can be added to the cell culture media.

In an example, the cell culture medium contains only human derived additives. Thus, in an example, the cell culture media is xeno-free. For avoidance of doubt, in these examples, the culture medium is free of animal proteins. In an example, cell culture medium used in the methods of the disclosure is free of animal components.

In an example, the culture medium comprises serum. In other examples the culture medium is fetal bovine serum free culture medium comprising growth factors that promote mesenchymal lineage precursor or stem cell proliferation. In an embodiment, the culture medium is serum free stem cell culture medium. In an example, the cell culture medium comprises:
a basal medium;
platelet derived growth factor (PDGF);
fibroblast growth factor 2 (FGF2).

In an example, the culture medium comprises platelet derived growth factor (PDGF) and fibroblast growth factor 2 (FGF2), wherein the level of FGF2 is less than about 6 ng/ml. For example, the FGF2 level may be less than about 5 ng/ml, less than about 4 ng/ml, less than about 3 ng/ml, less than about 2 ng/ml, less than about 1 ng/ml. In other examples, the FGF2 level is less than about 0.9 ng/ml, less than about 0.8 ng/ml, less than about 0.7 ng/ml, less than about 0.6 ng/ml, less than about 0.5 ng/ml, less than about 0.4 ng/ml, less than about 0.3 ng/ml, less than about 0.2 ng/ml.

In another example, the level of FGF2 is between about 1 pg/ml and 100 pg/ml. In another example, the level of FGF2 is between about 5 pg/ml and 80 pg/ml.

In an example, the PDGF is PDGF-BB. In an example, the level of PDGF-BB is between about 1 ng/ml and 150 ng/ml. In another example, the level of PDGF-BB is between about 7.5 ng/ml and 120 ng/ml. In another example, the level of PDGF-BB is between about 15 ng/ml and 60 ng/ml. In another example, the level of PDGF-BB is at least about 10 ng/ml. In another example, the level of PDGF-BB is at least about 15 ng/ml. In another example, the level of PDGF-BB is at least about 20 ng/ml. In another example, the level of PDGF-BB is at least about 21 ng/ml. In another example, the level of PDGF-BB is at least about 22 ng/ml. In another example, the level of PDGF-BB is at least about 23 ng/ml. In another example, the level of PDGF-BB is at least about 24 ng/ml. In another example, the level of PDGF-BB is at least about 25 ng/ml.

In another example, the PDGF is PDGF-AB. In an example, the level of PDGF-AB is between about 1 ng/ml and 150 ng/ml. In another example, the level of PDGF-AB is between about 7.5 ng/ml and 120 ng/ml. In another example, the level of PDGF-AB is between about 15 ng/ml and 60 ng/ml. In another example, the level of PDGF-AB is at least about 10 ng/ml. In another example, the level of PDGF-AB is at least about 15 ng/ml. In another example, the level of PDGF-AB is at least about 20 ng/ml. In another example, the level of PDGF-AB is at least about 21 ng/ml. In another example, the level of PDGF-AB is at least about 22 ng/ml. In another example, the level of PDGF-AB is at least about 23 ng/ml. In another example, the level of PDGF-AB is at least about 24 ng/ml. In another example, the level of PDGF-AB is at least about 25 ng/ml.

In other examples, additional factors can be added to the cell culture medium. In an example, the culture medium further comprising EGF. EGF is a growth factor that stimulates cell proliferation by binding to its receptor EGFR. In an example, the method of the present disclosure comprises culturing a population of stem cells in a fetal bovine serum free cell culture medium further comprising EGF. In an example, the level of EGF is between about 0.1 and 7 ng/ml. For example, the level of EGF can be at least about 5 ng/ml.

In another example, the level of EGF is between about 0.2 ng/ml and 3.2 ng/ml. In another example, the level of EGF is between about 0.4 ng/ml and 1.6 ng/ml. In another example, the level of EGF is between about 0.2 ng/ml. In another example, the level of EGF is at least about 0.3 ng/ml. In another example, the level of EGF is at least about 0.4 ng/ml. In another example, the level of EGF is at least about 0.5 ng/ml. In another example, the level of EGF is at least about 0.6 ng/ml. In another example, the level of EGF is at least about 0.7 ng/ml. In another example, the level of EGF is at least about 0.8 ng/ml. In another example, the level of EGF is at least about 0.9 ng/ml. In another example, the level of EGF is at least about 1.0 ng/ml.

In the above examples, basal medium such as Alpha MEM or StemSpan™ can be supplemented with the referenced quantity of growth factor. In an example, the culture medium comprises Alpha MEM or StemSpan™ supplemented with 32 ng/ml PDGF-BB, 0.8 ng/ml EGF and 0.02 ng/ml FGF.

In other examples, additional factors can be added to the cell culture medium. For example, the cell culture media can be supplemented with one or more stimulatory factors selected from the group consisting of epidermal growth factor (EGF), 1α,25-dihydroxyvitamin D3 (1,25D), tumor necrosis factor α (TNF-α), interleukin-1β (IL-1β) and stromal derived factor 1α (SDF-1α). In another embodiment, cells may also be cultured in the presence of at least one cytokine in an amount adequate to support growth of the cells. In another embodiment, cells can be cultured in the presence of heparin or a derivative thereof. For example, the cell culture medium may contain about 50 ng/ml of heparin. In other examples, the cell culture medium contains about 60 ng/ml of heparin, about 70 ng/ml of heparin, about 80 ng/ml of heparin, about 90 ng/ml of heparin, about 100 ng/ml of heparin, about 110 ng/ml of heparin, about 110 ng/ml of heparin, about 120 ng/ml of heparin, about 130 ng/ml of heparin, about 140 ng/ml of heparin, about 150 ng/ml of heparin or a derivative thereof. In an example, the heparin derivative is a sulphate). Various forms of heparin sulphate are known in the art and include heparin sulphate 2 (HS2). HS2 can be derived from various sources including for example, the liver of male and/or female mammals. Thus, an exemplary heparin sulphate includes male liver heparin sulphate (MML HS) and female liver heparin sulphate (FML HS).

In another example, the cell culture medium of the present disclosure promotes stem cell proliferation while maintaining stem cells in an undifferentiated state. Stem cells are considered to be undifferentiated when they have not committed to a specific differentiation lineage. As discussed above, stem cells display morphological characteristics that distinguish them from differentiated cells. Furthermore, undifferentiated stem cells express genes that may be used as markers to detect differentiation status. The polypeptide products may also be used as markers to detect differentiation status. Accordingly, one of skill in the art could readily determine whether the methods of the present disclosure maintain stem cells in an undifferentiated state using routine morphological, genetic and/or proteomic analysis.

### Modification of the Cells

The mesenchymal lineage precursor or stem cells disclosed herein may be altered in such a way that upon administration, lysis of the cell is inhibited. Alteration of an antigen can induce immunological non-responsiveness or tolerance, thereby preventing the induction of the effector phases of an immune response (e.g., cytotoxic T cell generation, antibody production etc.) which are ultimately responsible for rejection of foreign cells in a normal immune response. Antigens that can be altered to achieve this goal include, for example, MEW class I antigens, MEW class II antigens, LFA-3 and ICAM-1.

The mesenchymal lineage precursor or stem cells may also be genetically modified to express proteins of importance for the differentiation and/or maintenance of striated skeletal muscle cells. Exemplary proteins include growth factors (TGF-β, insulin-like growth factor 1 (IGF-1), FGF), myogenic factors (e.g. myoD, myogenin, myogenic factor 5 (Myf5), myogenic regulatory factor (MRF)), transcription factors (e.g. GATA-4), cytokines (e.g. cardiotropin-1), members of the neuregulin family (e.g. neuregulin 1, 2 and 3) and homeobox genes (e.g. Csx, tinman and NKx family).

### Compositions

Mesenchymal lineage or stem cells disclosed herein can be culture expanded from a cryopreserved intermediate to produce a preparation containing at least one therapeutic dose.

In an example, compositions of the disclosure are defined in terms of their expression of a receptor which activates NF-κB. In an example, NF-κB activation is measured by the level of NF-κB phosphorylation. Examples of receptors that activate NF-κB include TNF-R1 and IL-1R. Various means of measuring NF-κB activation are known in the art. For example, receptor mediated phosphorylation of NF-κB can be measured via ELISA or immunofluorescence after appropriate stimulation of cells. In an example, the level of NF-κB activation in stimulated MLPSCs is compared with the level of NF-κB activation in unstimulated MLPSCs. For example, MLPSCs may be stimulated by contact with TNF-α before the level of NF-κB activation is determined. The level of NF-κB activation in the stimulated cells is then compared with the level of NF-κB activation in MLPSCs that were not contacted with TNF-α.

In an example, MLPCs of the disclosure express a receptor which, upon activation via an inflammatory stimulus, phosphorylates NF-κB, wherein the MLPSCs express a level of the receptor that is sufficient to increase phosphorylation of NF-κB upon activation via the inflammatory stimulus at least 3.0 fold greater than unstimulated MLPSCs. In another example, the MLPSCs express a level of the receptor that is sufficient to increase phosphorylation of NF-κB upon activation via the inflammatory stimulus at least 3.5 fold greater than unstimulated MLPSCs. In another example, the MLPSCs express a level of the receptor that is sufficient to increase phosphorylation of NF-κB upon activation via the inflammatory stimulus at least 4 fold greater than unstimulated MLPSCs.

In an example, the receptor that increases phosphorylation of NF-κB upon activation via an inflammatory stimulus relative to unstimulated MLPSCs is TNF-R1.

Accordingly, in an example, compositions of the disclosure comprise cells that express high levels of TNF-R1 after being thawed and culture expanded. Accordingly, the present disclosure encompasses compositions which comprise a population of culture expanded mesenchymal lineage precursor or stem cells (MLPSC), wherein the population of MLPSCs are culture expanded from a cryopreserved intermediate MLPSC preparation and the culture expanded MLPSCs express specified levels of TNF-R1 under culture conditions. For example, the culture expanded population of MLPSCs can express about 200 pg/ml TNF-R1 under culture conditions. In another example, the mesenchymal lineage precursor or stem cells culture expanded from a cryopreserved intermediate express about 220 pg/ml TNF-R1 under culture conditions. In another example, the mesenchymal lineage precursor or stem cells culture expanded from a cryopreserved intermediate express about 225 pg/ml TNF-R1 under culture conditions. In another example, the mesenchymal lineage precursor or stem cells culture expanded from a cryopreserved intermediate express about 230 pg/ml TNF-R1 under culture conditions. In another example, the mesenchymal lineage precursor or stem cells culture expanded from a cryopreserved intermediate express about 245 pg/ml TNF-R1 under culture conditions.

In another example, the mesenchymal lineage precursor or stem cells culture expanded from a cryopreserved intermediate express about 250 pg/ml TNF-R1 under culture conditions. In another example, the mesenchymal lineage precursor or stem cells culture expanded from a cryopreserved intermediate express about 260 pg/ml TNF-R1 under culture conditions. In another example, the mesenchymal lineage precursor or stem cells culture expanded from a cryopreserved intermediate express about 270 pg/ml TNF-R1 under culture conditions. In another example, the mesenchymal lineage precursor or stem cells culture expanded from a cryopreserved intermediate express about 280 pg/ml TNF-R1 under culture conditions. In another example, the mesenchymal lineage precursor or stem cells culture expanded from a cryopreserved intermediate express about 290 pg/ml TNF-R1 under culture conditions. In another example, the mesenchymal lineage precursor or stem cells culture expanded from a cryopreserved intermediate express about 300 pg/ml TNF-R1 under culture conditions. In another example, the mesenchymal lineage precursor or stem cells culture expanded from a cryopreserved intermediate express between 200 pg/ml and 500 pg/ml TNF-R1 under culture conditions. In another example, the mesenchymal lineage precursor or stem cells culture expanded from a cryopreserved intermediate express between 220 pg/ml and 450 pg/ml TNF-R1 under culture conditions. In another example, the mesenchymal lineage precursor or stem cells culture expanded from a cryopreserved intermediate express between 225 pg/ml and 450 pg/ml TNF-R1 under culture conditions.

Similarly, in an example, the culture expanded population of MLPSCs can express about 23.5 pg/10⁶ cells TNF-R1 under culture conditions. In another example, the mesenchymal lineage precursor or stem cells culture expanded from a cryopreserved intermediate express about 26.5 pg TNF-R1 per 10⁶ cells TNF-R1 under culture conditions. In another example, the mesenchymal lineage precursor or stem cells culture expanded from a cryopreserved intermediate express about 29 pg TNF-R1 per 10⁶ cells TNF-R1 under culture conditions. In another example, the mesenchymal lineage precursor or stem cells culture expanded from a cryopreserved intermediate express about 30 pg TNF-R1 per 10⁶ cells TNF-R1 under culture conditions. In another example, the mesenchymal lineage precursor or stem cells culture expanded from a cryopreserved intermediate express about 32 pg TNF-R1 per 10⁶ cells TNF-R1 under culture conditions. In another example, the mesenchymal lineage precursor or stem cells culture expanded from a cryopreserved intermediate express about 33 pg TNF-R1 per 10⁶ cells TNF-R1 under culture conditions. In another example, the mesenchymal lineage precursor or stem cells culture expanded from a cryopreserved intermediate express about 34 pg TNF-R1 per 10⁶ cells TNF-R1 under culture conditions. In another example, the mesenchymal lineage precursor or stem cells culture expanded from a cryopreserved intermediate express about 35 pg TNF-R1 per 10⁶ cells TNF-R1 under culture conditions. In another example, the mesenchymal lineage precursor or stem cells culture expanded from a cryopreserved intermediate express between 27 pg TNF-R1 per 10⁶ cells and 59 pg TNF-R1 per 10⁶ cells TNF-R1 under culture conditions. In another example, the mesenchymal lineage precursor or stem cells culture expanded from a cryopreserved intermediate express between 29 pg TNF-R1 per 10⁶ cells and 53 pg TNF-R1 per 10⁶ cells TNF-R1 under culture conditions. In another example, the mesenchymal lineage precursor or stem cells culture expanded from a cryopreserved intermediate express between 32 pg TNF-R1 per 10⁶ cells and 53 pg TNF-R1 per 10⁶ cells TNF-R1 under culture conditions.

In an example, the population of culture expanded cells is selected for use in treatment of an inflammatory disease by determining expression of TNF-R1 under culture conditions. For example, expression of an above referenced level of TNF-R1 may be determined under culture conditions. In an example, the population of culture expanded cells is selected for use in treatment of graft versus host disease (GvHD). However, various other examples of inflammatory disorders are discussed herein.

In an example, compositions of the disclosure comprise MLPSCs which secrete elevated levels of one or more of MCP-1, M-CSF and PGE2 under culture conditions when exposed to inflammatory stimuli. In an example, the levels are elevated relative to unstimulated MLPSCs. In an example, MLPSCs of the disclosure secrete at least 16,000 pg/ml MCP-1 when exposed to inflammatory stimuli. In an example, MLPSCs of the disclosure secrete at least 18,000 pg/ml MCP-1 when exposed to inflammatory stimuli. In another example, MLPSCs of the disclosure secrete at least 1,200 pg/ml M-CSF when exposed to inflammatory stimuli. In another example, MLPSCs of the disclosure secrete at least 1,500 pg/ml M-CSF when exposed to inflammatory stimuli. In an example, the inflammatory stimuli is TNF-α. In another example, MLPSCs of the disclosure secrete at least 30,000 pg/ml PGE2 when exposed to inflammatory stimuli. In another example, MLPSCs of the disclosure secrete at least 40,000 pg/ml PGE2 when exposed to inflammatory stimuli. In an example, the inflammatory stimuli is TNF-α and/or IL-1β. In an example, the inflammatory stimuli is TNF-α and IL-1β.

In another example, compositions of the disclosure comprise cells that inhibit IL-2Rα expression. For example, compositions of the disclosure can comprise a population of culture expanded mesenchymal lineage precursor or stem cells (MLPSC), wherein the population of MLPSCs are culture expanded from a cryopreserved intermediate MLPSC preparation and the culture expanded MLPSCs inhibit IL-2Rα expression by at least 55% under culture conditions. In another example, the MLPSCs inhibit IL-2Rα expression by at least 58% under culture conditions. In another example, the MLPSCs inhibit IL-2Rα expression by at least 60% under culture conditions. In another example, the MLPSCs inhibit IL-2Rα expression between 55 and 75% under culture conditions. In another example, the MLPSCs inhibit IL-2Rα expression between 58 and 65% under culture conditions. In an example, such cells express an above referenced level of TNF-R1 under culture conditions.

In an example, compositions of the disclosure comprises at least 5×10⁶ cells. In another example, compositions comprises at least 10×10⁶ cells. In another example, compositions comprises at least 15×10⁶ cells. In another example, compositions comprises at least 20×10⁶ cells. In another example, compositions comprises at least 25×10⁶ cells. In another example, compositions comprises at least 30×10⁶ cells. In another example, compositions comprises at least 35×10⁶ cells. In another example, compositions comprises at least 40×10⁶ cells. In another example, compositions comprises at least 50×10⁶ cells. In another example, compositions comprises between 5×10⁶ cells and 50×10⁶ cells. In another example, compositions comprises between 10×10⁶ cells and 35×10⁶ cells. In another example, compositions comprises between 20×10⁶ cells and 30×10⁶ cells. In other examples, compositions comprise at least 100×10⁶ cells. In another example, compositions comprises between 50×10⁶ cells and 500×10⁶ cells.

In one example, compositions of the disclose comprise a pharmaceutically acceptable carrier and/or excipient. The terms “carrier” and “excipient” refer to compositions of matter that are conventionally used in the art to facilitate the storage, administration, and/or the biological activity of an active compound (see, e.g., Remington's Pharmaceutical Sciences, 16th Ed., Mac Publishing Company (1980). A carrier may also reduce any undesirable side effects of the active compound. A suitable carrier is, for example, stable, e.g., incapable of reacting with other ingredients in the carrier. In one example, the carrier does not produce significant local or systemic adverse effect in recipients at the dosages and concentrations employed for treatment.

Suitable carriers for the present disclosure include those conventionally used, e.g., water, saline, aqueous dextrose, lactose, Ringer's solution, a buffered solution, hyaluronan and glycols are exemplary liquid carriers, particularly (when isotonic) for solutions. Suitable pharmaceutical carriers and excipients include starch, cellulose, glucose, lactose, sucrose, gelatin, malt, rice, flour, chalk, silica gel, magnesium stearate, sodium stearate, glycerol monostearate, sodium chloride, glycerol, propylene glycol, water, ethanol, and the like.

In another example, a carrier is a media composition, e.g., in which a cell is grown or suspended. Such a media composition does not induce any adverse effects in a subject to whom it is administered. Exemplary carriers and excipients do not adversely affect the viability of a cell and/or the ability of a cell to treat or prevent disease.

In one example, the carrier or excipient provides a buffering activity to maintain the cells and/or soluble factors at a suitable pH to thereby exert a biological activity, e.g., the carrier or excipient is phosphate buffered saline (PBS). PBS represents an attractive carrier or excipient because it interacts with cells and factors minimally and permits rapid release of the cells and factors, in such a case, the composition of the disclosure may be produced as a liquid for direct application to the blood stream or into a tissue or a region surrounding or adjacent to a tissue, e.g., by injection.

Compositions of the disclosure may be cryopreserved. Cryopreservation of mesenchymal lineage precursor or stem cells can be carried out using slow-rate cooling methods or ‘fast’ freezing protocols known in the art. Preferably, the method of cryopreservation maintains similar phenotypes, cell surface markers and growth rates of cryopreserved cells in comparison with unfrozen cells.

The cryopreserved composition may comprise a cryopreservation solution. The pH of the cryopreservation solution is typically 6.5 to 8, preferably 7.4.

The cryopreservation solution may comprise a sterile, non-pyrogenic isotonic solution such as, for example, PlasmaLyte ATM. 100 mL of PlasmaLyte ATM contains 526 mg of sodium chloride, USP (NaCl); 502 mg of sodium gluconate (C6H11NaO7); 368 mg of sodium acetate trihydrate, USP (C2H3NaO2·3H2O); 37 mg of potassium chloride, USP (KCl); and 30 mg of magnesium chloride, USP (MgCl2·6H2O). It contains no antimicrobial agents. The pH is adjusted with sodium hydroxide. The pH is 7.4 (6.5 to 8.0).

The cryopreservation solution may comprise Profreeze™. The cryopreservation solution may additionally or alternatively comprise culture medium, for example, αMEM.

To facilitate freezing, a cryoprotectant such as, for example, dimethylsulfoxide (DMSO), is usually added to the cryopreservation solution. Ideally, the cryoprotectant should be nontoxic for cells and patients, nonantigenic, chemically inert, provide high survival rate after thawing and allow transplantation without washing. However, the most commonly used cryoprotector, DMSO, shows some cytotoxicity. Hydroxylethyl starch (HES) may be used as a substitute or in combination with DMSO to reduce cytotoxicity of the cryopreservation solution.

The cryopreservation solution may comprise one or more of DMSO, hydroxyethyl starch, human serum components and other protein bulking agents. In one example, the cryopreserved solution comprises Plasma-Lyte A (70%), DMSO (10%), HSA (25%) solution, the HSA solution comprising 5% HSA and 15% buffer.

In an example, the cryopreservation solution may further comprise one or more of methylcellulose, polyvinyl pyrrolidone (PVP) and trehalose.

The cryopreserved composition may be thawed and administered directly to the subject or added to another solution, for example, comprising hyaluronic acid. Alternatively, the cryopreserved composition may be thawed and the mesenchymal lineage precursor or stem cells resuspended in an alternate carrier prior to administration.

The compositions described herein may be administered alone or as admixtures with other cells. The cells of different types may be admixed with a composition of the disclosure immediately or shortly prior to administration, or they may be co-cultured together for a period of time prior to administration.

In one example, the composition comprises an effective amount or a therapeutically or prophylactically effective amount of mesenchymal lineage precursor or stem cells and/or progeny thereof and/or soluble factor derived therefrom. For example, the composition comprises about 1×10⁵ stem cells to about 1×10⁹ stem cells or about 1.25×10³ stem cells to about 1.25×10⁷ stem cells/kg (80 kg subject). The exact amount of cells to be administered is dependent upon a variety of factors, including the age, weight, and sex of the subject, and the extent and severity of the disorder being treated.

Despite the number of cells provided in the composition, in an example, 50×10⁶ to 200×10⁷ cells are administered. In other examples, 60×10⁶ to 200×10⁶ cells or 75×10⁶ to 150×10⁶ cells are administered. In an example, 75×10⁶ cells are administered. In another example, 150×10⁶ cells are administered.

In an example, the composition comprises greater than 5.00×10⁶ viable cells/mL. In another example, the composition comprises greater than 5.50×10⁶ viable cells/mL. In another example, the composition comprises greater than 6.00×10⁶ viable cells/mL. In another example, the composition comprises greater than 6.50×10⁶ viable cells/mL. In another example, the composition comprises greater than 6.68×10⁶ viable cells/mL.

In an example, the mesenchymal lineage precursor or stem cells comprise at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99% of the cell population of the composition.

In an example, the compositions described herein may be administered as a single dose. In another example, cellular compositions are administered over multiple doses. For example, at least 2, at least 3, at least 4, at least 5, at least 6, at least 7, at least 8, at least 9, at least 10 doses. For example, doses can be administered twice weekly.

In an example, the composition may optionally be packaged in a suitable container with written instructions for a desired purpose, such as mixing of the composition with cell culture media to provide a specific concentration.

In an example, the composition is provided in a bioreactor.

Compositions of the disclosure may be administered systemically, such as, for example, by intravenous, intraarterial, or intraperitoneal administration. In other examples, compositions may be administered by intranasal, intramuscular or intracardiac administration. In other examples, compositions of the disclosure may be administered to the subjects airway. For example, the composition may be administered to the lung(s) of a subject. In another example, compositions are administered intravenously and to the subjects airway.

### Determining the Amount of Receptor Expression

The level of receptor (e.g. TNF-R1 or IL-2Rα) expressed from cells can be determined via various assays known in the art. Examples include Western blot, enzyme-linked immunosorbent assay (ELISA), fluorescence-linked immunosorbent assay (FLISA), competition assay, radioimmunoassay, lateral flow immunoassay, flow-through immunoassay, electrochemiluminescent assay, nephelometric-based assays, turbidimetric-based assay, fluorescence activated cell sorting (FACS)-based assays for detection of TGFβ1 in culture medium used to culture mesenchymal lineage or precursor cells, and surface plasmon resonance (SPR or Biacore).

One form of a suitable assay is, for example, an ELISA or FLISA. In this example, such an assay involves immobilizing a TNF-R1 binding protein onto a solid matrix, such as, for example a polystyrene or polycarbonate microwell or dipstick, a membrane, or a glass support (e.g., a glass slide). A test sample such as a sample of cell culture media from cells in exponential growth phase is then brought into direct contact with the TNF-R1 binding protein and TNF-R1 in the sample is bound or captured. Following washing to remove any unbound protein in the sample, a protein that binds to TNF-R1 at a distinct epitope is brought into direct contact with the captured TNF-R1. This detector protein is generally labelled with a detectable reporter molecule, such as, for example, an enzyme (e.g. horseradish peroxidase (HRP)), alkaline phosphatase (AP) or β-galactosidase) in the case of an ELISA or a fluorophore in the case of a FLISA. Alternatively, a second labeled protein can be used that binds to the detector protein. Following washing to remove any unbound protein the detectable reporter molecule is detected by the addition of a substrate in the case of an ELISA, such as, for example, hydrogen peroxide, TMB, or toluidine, or 5-bromo-4-chloro-3-indol-beta-D-galactopyranoside (x-gal). The immobilized (capture) protein and the detector protein may be used in the opposite manner.

The level of the antigen in the sample is then determined using a standard curve that has been produced using known quantities of the marker or by comparison to a control sample. In an example, the level of antigen is compared to the number of cells in the analysed sample. For example, the level of antigen may be presented relative to 10⁶ cells.

The assays described above are readily modified to use chemiluminescence or electrochemiluminescence as the basis for detection. The assays described above are also readily modified to determine levels of other markers such as IL-2Rα.

As will be apparent to the skilled person, other detection methods based on an immunosorbent assay are useful in the performance of the present disclosure. For example, an immunosorbent method based on the description above using a radiolabel for detection, or a gold label (e.g., colloidal gold) for detection, or a liposome, for example, encapsulating NAD+ for detection or an acridinium linked immunosorbent assay.

In some examples of the disclosure, the level of TGFβ1 is determined using a surface plasmon resonance detector (e.g., BIAcore™, GE Healthcare, Piscataway, N.J.), a flow through device (e.g., as described in U.S. Pat. No. 7,205,159), a micro- or nano-immunoassay device (e.g., as described in U.S. Pat. No. 7,271,007), a lateral flow device (e.g., as described in US publication 20040228761 or US publication 20040265926), a fluorescence polarization immunoassay (FPIA, e.g., as described in U.S. Pat. No. 4,593,089 or U.S. Pat. No. 4,751,190), or an immunoturbidimetric assay (e.g., as described in U.S. Pat. No. 5,571,728 or U.S. Pat. No. 6,248,597).

### Potency Assay

In an example, the present disclosure encompasses a method for determining the therapeutic efficacy of mesenchymal lineage precursor or stem cells comprising:
(i) obtaining a population comprising mesenchymal lineage precursor or stem cells;
(ii) culturing the cells in a culture medium; and
(iii) determining the amount of TNF-R1 expressed by the cells into the culture medium under culture conditions.

In an example, an amount of at least about 200 pg/ml [23.5 pg/10⁶ cells] TNF-R1 is indicative of therapeutic efficacy. In an example, an amount of at least about 225 pg/ml [26.5 pg/10⁶ cells] TNF-R1 is indicative of therapeutic efficacy. In another example, at least about 230 pg/ml TNF-R1 is indicative of therapeutic efficacy. In another example, at least about 250 pg/ml TNF-R1 is indicative of therapeutic efficacy. In another example, at least about 260 pg/ml TNF-R1 is indicative of therapeutic efficacy. In another example, the at least about 270 pg/ml TNF-R1 is indicative of therapeutic efficacy. In another example, at least about 280 pg/ml TNF-R1 is indicative of therapeutic efficacy. In another example, at least about 290 pg/ml TNF-R1 is indicative of therapeutic efficacy. In another example, at least about 300 pg/ml TNF-R1 is indicative of therapeutic efficacy. In another example, between 200 pg/ml and 500 pg/ml TNF-R1 is indicative of therapeutic efficacy. In another example, between 220 pg/ml and 450 pg/ml TNF-R1 is indicative of therapeutic efficacy. In another example, between 225 pg/ml and 450 pg/ml TNF-R1 is indicative of therapeutic efficacy. In another example, a corresponding amount of TNF-R1 in pg/10⁶ cells is indicative of therapeutic efficacy. In another example, between 230 pg/ml and 450 pg/ml TNF-R1 is indicative of therapeutic efficacy. In another example, a corresponding amount of TNF-R1 in pg/10⁶ cells is indicative of therapeutic efficacy.

In another example, at least about 55% inhibition of IL-2Rα expression is indicative of therapeutic efficacy. In another example, at least about 58% inhibition of IL-2Rα expression is indicative of therapeutic efficacy. In another example, at least about 60% inhibition of IL-2Rα expression is indicative of therapeutic efficacy. In another example, between 55% and 75% inhibition of IL-2Rα expression is indicative of therapeutic efficacy. In another example, between 58% and 65% inhibition of IL-2Rα expression is indicative of therapeutic efficacy.

In an example, the present disclosure encompasses a method for determining the therapeutic efficacy of mesenchymal lineage precursor or stem cells comprising:
(i) obtaining a population comprising mesenchymal lineage precursor or stem cells;
(ii) culturing the cells in a culture medium; and
(iii) determining the amount of IL-2Rα inhibition by the cells under culture conditions. In an example, at least about 55% inhibition of IL-2Rα expression is indicative of therapeutic efficacy. In another example, at least about 58% inhibition of IL-2Rα expression is indicative of therapeutic efficacy. In another example, at least about 60% inhibition of IL-2Rα expression is indicative of therapeutic efficacy. In another example, between 55% and 75% inhibition of IL-2Rα expression is indicative of therapeutic efficacy. In another example, between 58% and 65% inhibition of IL-2Rα expression is indicative of therapeutic efficacy.

In an example, the population of mesenchymal lineage precursor or stem cells is obtained from a 3D cell culture. For example, the population can be obtained from a bioreactor.

In an example, the population of mesenchymal lineage precursor or stem cells are culture expanded in 3D cell culture from a cryopreserved intermediate MLPSC preparation. In an example, the level of TNF-R1 is determined before the cells are cryopreserved.

### Treatment

In an example, the present disclosure encompasses a methods of treating a subject with an inflammatory disease. In an example, the inflammatory disease is a T cell mediated inflammatory disease.

In an example, the inflammatory disease is graft versus host disease. “Graft versus Host Disease (GvHD)” is an immunological disorder that is the major factor that limits the success and availability of allogeneic bone marrow or stem cell transplantation. GvHD occurs in acute (aGvHD) or chronic (cGvHD) forms. Acute GvHD usually manifests within 100 days following bone marrow or stem cell transplantation. Chronic GvHD generally manifests later than aGvHD (>100 days post transplantation) and has some features of autoimmune diseases. It may develop either de novo, following resolution of aGvHD or as an extension of aGvHD. Chronic GvHD can cause multiple, often debilitating symptoms, including widespread skin rashes, painful mouth ulcers, shortness of breath, and limb and joint pain. In an example, patients with cGvHD have impaired reconstitution of CD5+ B cells. In an example, cGvHD is refractory to steroid therapy. In an example, cGvHD is refractory to a biologic therapy. In an example cGvHD is refractory to steroid therapy and a biologic therapy.

In example, the inflammatory disease is inflammatory bowel disease (IBD). In an example, the inflammatory disease is Crohn's disease. In an example, the inflammatory disease is ulcerative colitis. In an example, compositions of the disclosure are administered to limit inflammation in the gut.

In an example, the inflammatory disease is hyperinflammation. In an example, the hyperinflammation is caused by a viral infection. The viral infection may be caused, for example, by a rhinovirus, influenza virus, respiratory syncytial virus (RSV) or a coronavirus. In an example, the hyperinflammation is caused by a coronavirus infection. The coronavirus may be Severe Acute Respiratory Syndrome coronavirus (SARS-CoV), Middle East Respiratory Syndrome coronavirus (MERS-CoV), COVID-19, 229E, NL63, OC43, or KHU1. In one example, the coronavirus is SARS-CoV, MERS-CoV or COVID-19. In an example, the subject also has Acute Respiratory Distress Syndrome (ARDS). In an example, the hyperinflammation is caused by cytokine storm.

In an example, the inflammatory disease is arthritis. In an example, the arthritis is osteoarthritis or rheumatoid arthritis. In an example, compositions of the disclosure are administered to reduce inflammation in bone.

In another example, the inflammatory disease is diabetes or an associated condition or symptom of diabetes selected from the group consisting of abnormal wound healing, symptoms of a heart attack, symptoms of a stroke, symptoms of peripheral vascular disease, amputation, symptoms of kidney disease, kidney failure, blindness, neuropathy, nephropathy, retinopathy, inflammation, impotence or nonalcoholic steatohepatitis (NASH).

In another example, the inflammatory disease is diabetic retinopathy.

Other examples of inflammatory diseases treated according to the disclosure include, pruritus, skin inflammation, psoriasis, multiple sclerosis, systemic lupus erythematosus, Hashimoto's thyroids, myasthenia gravis, diabetic nephropathy, asthma, inflammatory lung injury, inflammatory liver injury, inflammatory glomerular injury, atopic dermatitis, allergic contact dermatitis, irritant contact dermatitis, seborrhoeic dermatitis, Sjoegren's syndrome.

In an example, the methods of the present disclosure encompass administering a total dose of 600 million cells. For example, a subject treated according to the present disclosure can receive multiple doses of an above referenced composition so long as the total dose of cells does not exceed 600 million cells. For example, the subject may receive 3 doses of 200 million cells. In an example, the total dose of cells is 500 million cells. In an example, the total dose of cells is 400 million cells. For example, the subject may receive 4 doses of 100 million cells. In an example, the subject receives 1 dose of 100 million cells at baseline followed by three doses of 100 million cells administered one per month over three months.

In an example, the inflammatory disease is inflammatory bowel disease. In an example, the inflammatory disease is ulcerative colitis (UC), irritable bowel syndrome, irritable colon syndrome, Crohn's colitis or Crohn's disease (CD).

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

This application claims priority from AU2020902827 filed on 10 Aug. 2020 the disclosures of which are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### EXAMPLES

### Example 1: Anti-Inflammatory Effects of Culture Expanded Mesenchymal Precursor Cells (MSCs)

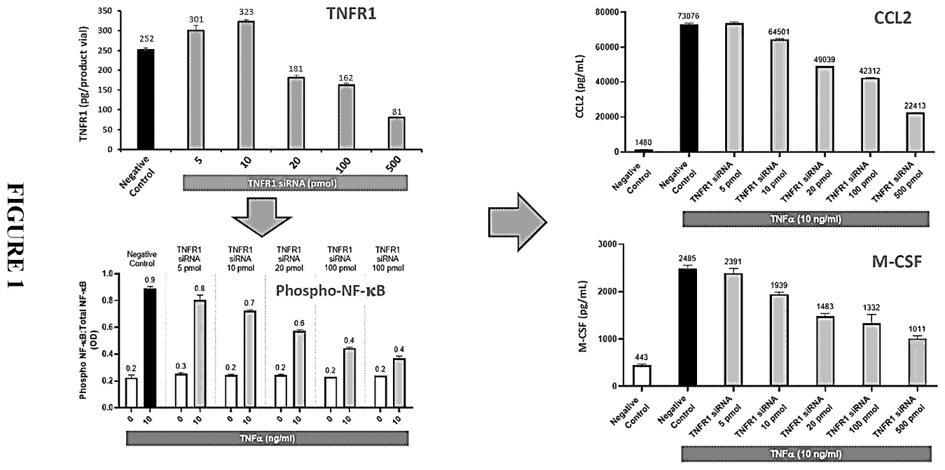

Culture expanded MSCs were transfected with siRNA targeting TNF-R1 (5, 10, 20, 100 or 500 pM) for 4 h. Medium was refreshed, and cells were cultured for 72 h. Cells were lysed and TNF-R1 levels were measured by ELISA. Samples were assayed in triplicate. In comparison to transfection with non-targeting siRNA control, transfection of MSCs with siRNA targeting TNF-R1 resulted in reduction of TNF-R1 expression by 30% (20 pM siRNA), 35% (100 pM) and 70% (500 pM) (FIG. 1).

Culture expanded MSCs were again transfected with siRNA targeting TNF-R1 (5, 10, 20, 100 or 500 pM). 48 h following transfection, cells were stimulated with TNFα (10 ng/ml) for 1 h then lysed. Levels of total and phosphorylated NF-kB in MSC lysates were determined by ELISA. siRNA-mediated reduction in the expression of TNF-R1 in MSCs resulted in a dose-dependent reduction in the level of the phosphorylated (ser-536) form of NF-kB (FIG. 1). Importantly, these data demonstrated that relatively small reductions in the level of TNF-R1 expressed by MSCs impact response of the cells to a maximal stimulus of TNF-alpha resulting in significantly impaired phosphorylation of NF-kB.

To examine whether TNF-alpha induces production of anti-inflammatory mediators by culture expanded MSCs which are capable of self-regulation and inhibition of TNF-alpha production, gene products whose expression is reported to be stimulated in MSC in response to exposure to TNF-alpha and have been implicated in immunomodulatory effects of MSC were assessed. These gene products included MCP-1/CCL2, M-CSF PGE2, all activated via the canonical NF-kB pathway.

For measurement of MCP-1, M-CSF and PGE2, MSCs were seeded in serum-supplemented medium in 24 well culture plates and allowed to attach overnight. Medium was refreshed and cells were incubated for 72 h in the presence or absence of TNFα (10 ng/ml). At the end of the culture period, conditioned medium was collected for analysis by ELISA.

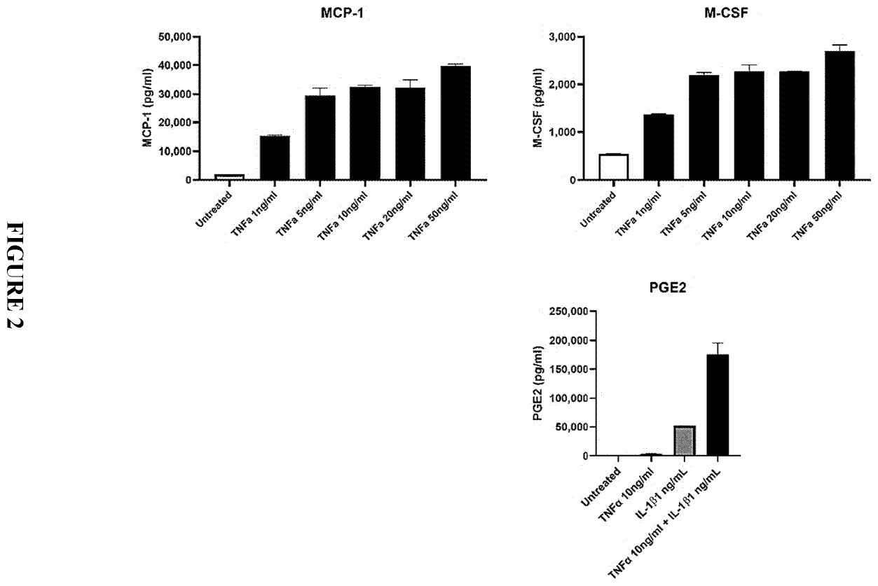

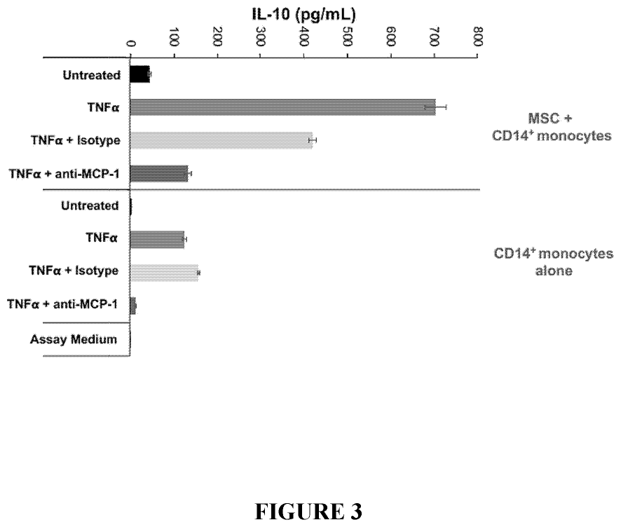

FIG. 1 shows that the reduction in phosphorylation of NF-kB is precisely mirrored by corresponding reductions in the levels of CCL2 and M-CSF secreted in the conditioned medium (FIG. 1). As shown in FIG. 2, TNF-alpha, in a dose-dependent manner, induced MSCs to secrete progressively higher levels of MCP-1/CCL2, M-CSF and PGE2. MCP-1 and M-CSF are secreted extracellularly, bind to their respective receptors on monocytes/macrophages and contribute to polarization of M1 macrophages to the resolving M2 phenotype which is associated with reduction in TNF-alpha production and increased production of anti-inflammatory cytokine IL-10. Indeed, as shown in FIG. 3, antibody mediated inhibition of MCP-1/CCL2 significantly impairs the secretion of IL-10 by CD14+ monocytes in co-culture with MSCs and TNF-alpha.

Together, these data confirm that culture expanded MSCs mediate anti-inflammatory effects due to secretion of soluble factors acting either directly on T cells, such as PGE2, or indirectly, such as MCP-1 or M-CSF, to induce M1 to M2 macrophage polarization and IL-10 secretion.

MSCs express receptors for a range of pro-inflammatory cytokines (Pittenger et al. (1999) Science, 284:143-147), suggesting that functional cell surface receptors may facilitate the immunomodulatory responses of culture expanded MSC to their environment, particularly in response to high levels of inflammatory cytokines such as TNFα as is observed in various inflammatory settings such as graft versus host disease or cytokine storm. The above findings suggest that the immunomodulatory effects of MSC in response to TNFα occur via TNF-R1 activation, NF-kB nuclear translocation, and transcription of multiple paracrine factors whose effects in concert result in both macrophage M2 polarization and T cell inhibition. These data suggest that MSCs expressing high levels of receptor(s) which mediate NF-kB activation such as TNF-R1 are advantageous for treating inflammatory disorders as, in response to inflammatory settings, there is increased capacity to direct both improved macrophage M2 polarization and T cell inhibition via NF-kB mediated transcription of multiple paracrine factors.

### Example 2: Potency Assay

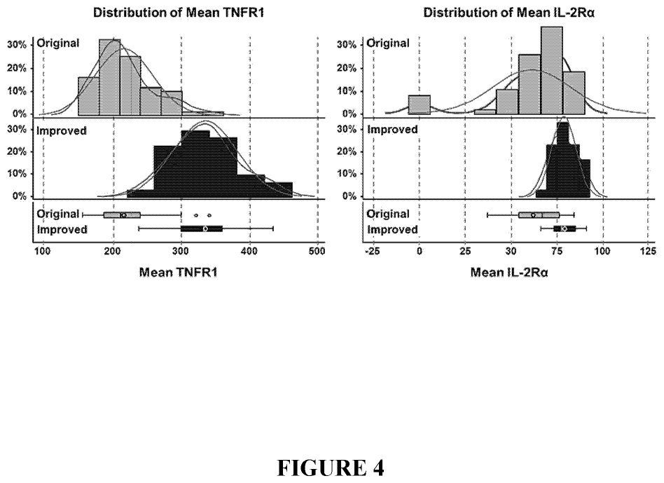

Surface expression of TNF-R1 on mesenchymal stem cells (MSC) was investigated following 3 clinical trials in steroid refractory (SR) graft versus host disease (GvHD) patients (trials 275, 280, 001/002; Table 1). Surface expression of TNF-R1 on MSC was intimately related to the ability of MSC to inhibit T cell proliferation as measured by inhibition of IL2-Rα expression (data not shown) with less variability observed at higher levels of TNF-R1 (FIG. 4). Furthermore, a significant correlation was observed between increasing TNF-R1 levels and increased inhibition of IL2-Rα:
TNF-R1>100 pg/ml, r²=0.13, p<0.0001
TNF-R1>190 pg/ml, r²=0.20, p<0.0001
TNF-R1>200 pg/ml, r²=0.22, p<0.0001
TNF-R1>210 pg/ml, r²=0.21, p<0.0001
TNF-R1>220 pg/ml, r²=0.17, p<0.0001
TNF-R1>250 pg/ml, r²=0.13, p<0.0001

The optimal TNF-R1 threshold to detect strongest correlation with IL2-Rα inhibition appeared to be a level above 200 pg/ml. The above findings support a rationale to set a TNF-R1 threshold, suggesting a level that best predicts secretion of paracrine factors which inhibit T cell activation/proliferation.

Based on these data, the objective was to validate two proposed potency assays 1) TNF-R1; and, 2) IL-2 Rα. The gold standard for potency validation is to determine a relationship to a clinical outcome.

The proposed potency assays were evaluated relative to overall survival in patients with acute GVHD who received mesenchymal stem cell therapy via randomised, controlled, open label trials referred to below as studies 265, 275, 280, 001/002 (Table 1):

TABLE 1
Overview of studies 265, 280, 001/002.

| Study 265 | Study 275 | Study 280 | Studies 001/002 |
|---|---|---|---|
| Patients with new | Patients with SR- | Patients with SR- | Patients with SR- |
| onset disease | aGVHD | aGVHD | aGVHD |
| | | Add-on therapy to | Initial second-line |
| | | institutional standard | therapy |
| Primary endpoint: | Primary endpoint: | Primary endpoint: | Primary Endpoint: |
| Treatment success | Overall Response | Durable Clinical | Day 28 OR, Day 100 |
| (multiple endpoints) | (CR + PR Day 28) | Response (CR ≥ 28 D | OS, and Day 180 OS |
| | | w/in 100 D) | |
| Predominantly non- | 81% Grade C/D | Less severe disease | 89% Grade C/D |
| severe disease | sever disease | 24% Grade B | severe disease |
| 46% Grade B | | 19% Skin-only | 11% Grade B |
| 38% Skin-only | | (includes Grade B) | 26% Skin-only |
| (includes Grade B) | | | (excludes Grade B) |
| Remestemcel-L | Day 28: OR 73% | Higher % Grade D in | Day 28 OR: 70% |
| treatment success | Day 100 OS: 66% | remestemecel-L arm | Day 100 OS:75% |
| similar to steroids | | Post hoc analysis of | Day 180 OS: 69% |
| | | Day 28 OR showed | |
| | | clinically meaningful | |
| | | efficacy in severe | |
| | | disease Grades C/D | |

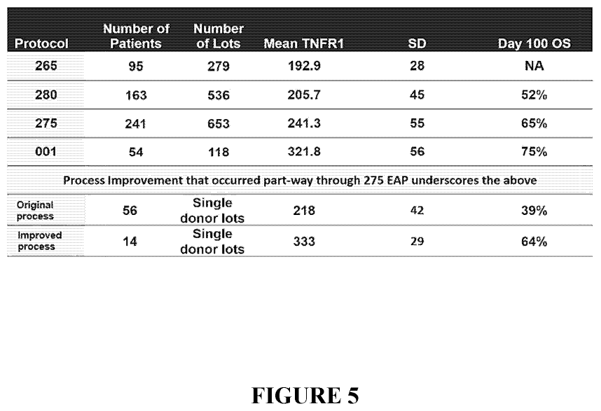

Surprisingly, logistic regression results for Day 100 OS were significant for TNF-R1 (p=0.002; 1.011 (1.004, 1.019); Point Estimate (95% CI); FIG. 5). Therefore, TNF-R1 appears to be a potency assay that is validated on the basis of correlation with Day 100 overall survival. These data provide sound basis for an improved composition for treatment of GvHD having a threshold level of TNF-R1 expression, in particular TNF-R1 greater than 200 pg/ml, more preferably greater than 225 pg/ml TNF-R1 under culture conditions.

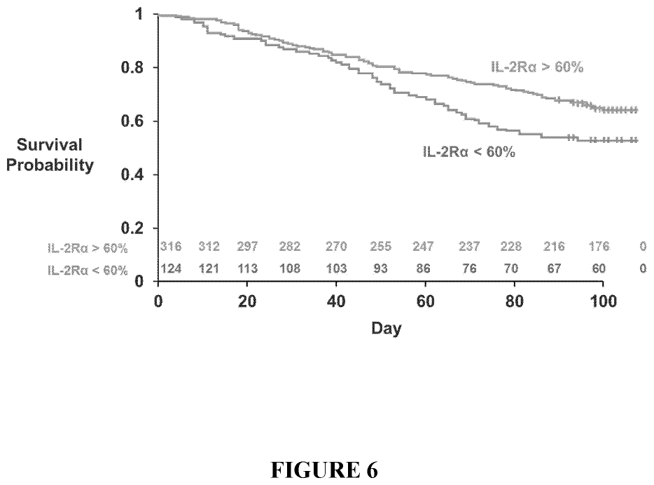

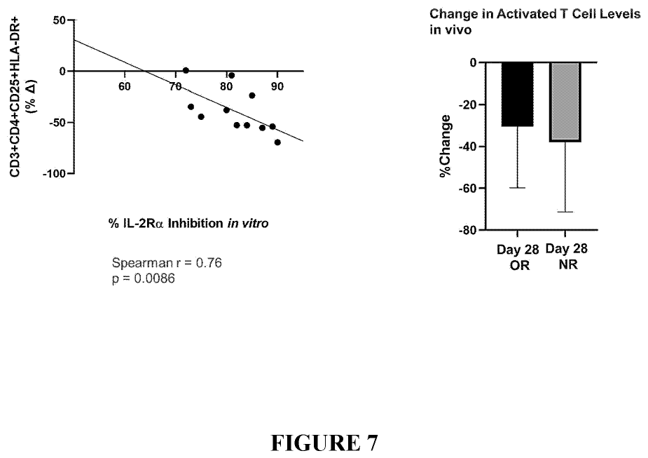

Significantly improved survival was also observed in GvHD patients that received MSCs with IL2-Rα expression levels >60% (FIG. 6). Furthermore, IL-2Rα inhibition correlated with in vivo reduction of immune activation (FIG. 7). FIG. 7 shows that there is the correlation between in vitro measurement of IL2-Rα inhibition and reduction in activated CD4+ T cells in vivo after 28 days of treatment with MSCs, in GvHD patients. The observed inverse relationship is significant using either the Spearman or Hoeffding coefficient measurements. The change in activated CD4+ T cell levels was seen in all patents irrespective of response status, indicating IL2-Rα inhibition is also a consistent in vivo measurement of MSC bioactivity.

### Example 3: Improved Manufacturing Method

Mesenchymal stem cells (MSC)s were defrosted from a cryopreserved intermediate population of MSCs and established in culture before being culture expanded in cell factories and then a bioreactor using a streamlined manufacturing process that reduces cell handling. Cells were culture expanded without CO₂ priming of cell factories and cytomate was eliminated from the culture process after passage 3. Removing CO₂ priming from the culture process reduced handling of cell factories and elimination of cytomate from passage 3 onwards required fewer sterile washes of the cells to complete a passage step. Furthermore, cells were contacted with trypsin for a minimum of 15 minutes when passaged, minimising handling of culture vessels.

TNF-R1 expression typically decreases significantly following cryopreservation of cells. However, TNF-R1 expression analysis of MSCs culture expanded from a cryopreserved intermediate via the improved manufacturing process with reduced cell handling surprisingly revealed that the cells expressed high levels of TNF-R1. As shown in FIG. 5, the cells express higher levels than previously produced cells (333 pg/ml v 218 pg/ml).

Next, clinical data was interrogated to examine directly whether there was a relationship between survival and whether patients received product made with cells culture expanded using previous techniques (“original manufacturing process”) or the improved manufacturing process discussed above. As shown below in Table 2, MSCs produced via the improved manufacturing process expressed significantly higher levels of TNF-R1 and significantly increased IL2-Rα inhibition. Furthermore, patients dosed with these MSCs had elevated 28 day overall response (OR) and significantly higher prospects of 100 day overall survival (OS).

TABLE 2
Higher levels of TNF-R1, IL2-Rα inhibition
and day 100 survival in patients treated with MSCs
produced using the improved manufacturing process.

| | TNFR1 (SD) | IL-2Ra (SD) | Day 28 | Day 100 |
|---|---|---|---|---|
| All Patients | (pg/mL) | (% inhibition) | OR | OS |
| Only Original | 213 (32) | 56 (25) | 63% | 58% |
| process (N = 348) | | | | |
| Only Improved | 328 (39) | 79 (6) | 70% | 75% |
| process (n = 92) | | | | |
| P-value | <0.0001 | <0.0001 | 0.2643 | 0.0026 |

*p-value for mean TNF-R1 from t-test;
p-value for 100 day OS from Fisher's Exact test

Taken together with the above findings from Examples 1 and 2, the present inventors have identified a population of MLPSCs with improved therapeutic efficacy, in particular MLPSCs that express at least 200 pg/ml TNF-R1, preferably 225 pg/ml and have provided a method of producing such cells, in particular MLPSCs expressing levels of TNF-R1 exceeding 331 pg/ml.

1. A composition comprising a population of culture expanded mesenchymal lineage precursor or stem cells (MLPSC), wherein the population of MLPSCs are culture expanded from a cryopreserved intermediate MLPSC preparation and the culture expanded MLPSCs express a receptor which, upon activation via an inflammatory stimulus, phosphorylates NF-κB, wherein the MLPSCs express a level of the receptor that is sufficient to increase phosphorylation of NF-κB upon activation via the inflammatory stimulus at least 3.5 fold greater than unstimulated MLPSCs.

2. (canceled)

3. The composition according to claim 1, wherein the receptor is one or both of TNF-R1 or IL-1R.

4. The composition according to claim 1, wherein activation of the MLPSCs via the inflammatory stimulus increases secretion of one or more of MCP-1, M-CSF and PGE2 under culture conditions.

5. (canceled)

6. The composition according to claim 1, wherein the receptor which, upon activation via an inflammatory stimulus, phosphorylates NF-κB is TNF-R1 and the MLPSCs express at least about 100 pg/ml to about 225 pg/ml TNF-R1 under culture conditions.

7. The composition according to claim 1, wherein the composition comprises at least 25×10⁶ cells.

8-9. (canceled)

10. A composition comprising a population of culture expanded mesenchymal lineage precursor or stem cells, wherein the composition comprises at least 25×10⁶ cells and, wherein the population of culture expanded cells has been selected for use in treatment of an inflammatory disease by determining expression of least about 200 pg/ml TNF-R1 under culture conditions.

11. The composition according to claim 10, wherein the population of culture expanded cells express at least about 100 pg/ml TNF-R1 to about 270 pg/ml TNF-R1 under culture conditions.

12-13. (canceled)

14. The composition according to claim 10, wherein the inflammatory disease is graft versus host disease (GvHD).

15-16. (canceled)

17. A method for determining the therapeutic efficacy of mesenchymal lineage precursor or stem cells comprising:
(i) obtaining a population comprising mesenchymal lineage precursor or stem cells;
(ii) culturing the cells in a culture medium; and
(iii) determining the amount of TNF-R1 expressed by the cells into the culture medium under culture conditions, wherein an amount of at least about 100 pg/ml TNF-R1 is indicative of therapeutic efficacy.

18-19. (canceled)

20. The method according to claim 17, wherein the population of mesenchymal lineage precursor or stem cells is obtained from a 3D cell culture.

21. (canceled)

22. A method of treating a subject with an inflammatory disease, the method comprising administering to a subject in need thereof a composition of mesenchymal lineage precursor or stem cells identified according to the method of claim 17.

23. (canceled)

24. The method according to claim 22, wherein the inflammatory disease is a T cell mediated inflammatory disease.

25. The method according to claim 22, wherein the inflammatory disease is GvHD.

26. (canceled)

27. The method according to claim 22, wherein a treated subject has improved 100 day survival.

28. The method according to claim 22, wherein the subject is refractory to steroid immunosuppressant and/or a biologic therapy.

29. The method according to claim 22, wherein the subject receives at least two doses of the composition.

30. The method according to claim 22, wherein the mesenchymal precursor lineage or stem cells are culture expanded from a cryopreserved intermediate MLPSC preparation.

31. The method according to claim 22, wherein the mesenchymal precursor lineage or stem cells are mesenchymal stem cells.

32. (canceled)

33. The method according to claim 22, wherein the composition further comprises Plasma-Lyte A (70%), DMSO (10%), HSA (25%) solution, the HSA solution comprising 5% HSA and 15% buffer.

34. The method according to claim 22, wherein the composition comprises greater than 6.68×10⁶ viable cells/mL.

35-36. (canceled)
